(19) United States
(12) Patent Application Publication
(10) Pub. No.: US 2020/0392001 A1
(43) Pub. Date: Dec. 17, 2020

(54) HYDROXYAPATITE BASED COMPOSITION AND FILM THEREOF COMPRISING INORGANIC FULLERENE-LIKE NANOPARTICLES OR INORGANIC NANOTUBES

(71) Applicants: YEDA RESEARCH AND DEVELOPMENT CO. LTD., Rehovot (IL); HOLON ACADEMIC INSTITUTE OF TECHNOLOGY, Holon (IL)

(72) Inventors: Reshef TENNE, Rehovot (IL); Hila SHALOM, Rehovot (IL); Lev RAPOPORT, Lod (IL)

(73) Assignees: YEDA RESEARCH AND DEVELOPMENT CO. LTD., Rehovot (IL); HOLON ACADEMIC INSTITUTE OF TECHNOLOGY, Holon (IL)

(21) Appl. No.: 16/970,958

(22) PCT Filed: Feb. 21, 2019
(86) PCT No.: PCT/IL2019/050203

§ 371 Date: Aug. 19, 2020

(30) Foreign Application Priority Data
Feb. 22, 2018 (IL) 257697

(51) Int. Cl. C01B 25/32; C25D 9/12; A61L 27/32; A61L 27/30; A61L 27/06; C01G 41/00; C01G 39/06; C01F 11/02

(52) U.S. Cl. CPC C01B 25/32; C25D 9/12; A61L 27/32; A61L 27/303; A61L 27/06; C01G 41/00; C01P 2004/64; C01F 11/02; A61L 2400/12; A61L 2420/04; A61L 2430/02; C01P 2004/13; C01G 39/06

(57) ABSTRACT

This invention is directed to compositions and films comprising hydroxyapatite with minute amounts of doped inorganic fullerene-like (IF) nanoparticles or doped inorganic nanotubes (INT); methods of preparation and uses thereof.

### FIELD OF THE INVENTION

This invention is directed to compositions and films comprising hydroxyapatite with minute amounts of doped inorganic fullerene-like (IF) nanoparticles or doped inorganic nanotubes (INT); methods of preparation and uses thereof.

### BACKGROUND OF THE INVENTION

Self-lubricating solid-state films are used for a variety of applications including the automotive, medical devices, power generation, machining, shipping, aerospace industries as well as many others. Often such films are a nanocomposite made of hard matrix containing a minority phase of a soft metal like copper or silver, or impregnated nanoparticles with good tribological performance [Basnyat, P.; et al. Mechanical and tribological properties of CrAlN—Ag self-lubricating films. Surf Coat. Technol. 2007, 202, 1011-1016].

More recently, self-lubricating films containing carbon nanotubes [Moghadam, A. D.; et al. Mechanical and tribological properties of self-lubricating metal matrix nanocomposites reinforced by carbon nanotubes (CNTs) and graphene-A review. Compos. Part B 2015, 77, 402-420], MoS₂ [Liu, E. Y.; Wang, W. Z.; Gao, Y. M.; Jia, J. H. Tribological properties of Ni-based self-lubricating composites with addition of silver and molybdenum disulfide. Tribol. Int. 2013, 57, 235-241.] and WS₂ nanoparticles [Lian, Y.; et al. Friction and wear behavior of WS₂/Zr self-lubricating soft coatings in dry sliding against 40Cr-hardened steel balls. Tribol. Lett. 2014, 53, 237-246] have been described.

Hydroxyapatite (HA, Ca₁₀(PO₄)₆(OH)₂) is used as a bone replacement material in a variety of orthopedic implants and artificial prostheses [Petit, R. The use of hydroxyapatite in orthopaedic surgery: A ten-year review. Eur. J. Orthop. Surg. Traumatol. 1999, 9, 71-74]. Given the fact that already 15% of the population is above 65 and increasing, artificial orthopedic implants have become a major health issue. However, this material suffers from high wear and poor fracture toughness. To alleviate these problems various methods were conceived including incorporation of nanoparticles (NP) into the HA films. In particular, HA films containing carbon [Lahiri, D.; et al. Carbon nanotube toughened hydroxyapatite by spark plasma sintering: Microstructural evolution and multiscale tribological properties. Carbon 2010, 48, 3103-3120] and boron nitride nanotubes [Lahiri, D.; et al. Boron nitride nanotube reinforced hydroxyapatite composite: Mechanical and tribological performance and in-vitro biocompatibility to osteoblasts. J. Mech. Behav. Biomed. 2011, 4, 44-56] were prepared by spark plasma sintering technique.

Frequently, HA phase also contains associated minerals and materials, including brushite and portlandite. Brushite—(CaH(PO₄)·2H₂O) is a metastable compound in physiological conditions and therefore it transforms into hydroxyapatite after implantation of a prostheses [Theiss, F.; Apelt, D.; Brand, B.; Kutter, A.; Zlinszky, K.; Bohner, M. Biocompatibility and resorption of a brushite calcium phosphate cement. Biomaterials 2005, 26, 4383-4394].

HA is synthesized in a hydrothermal reaction of CaO and monetite (CaHPO₄). High concentration of calcium oxide in the reaction leads to the formation of excess portlandite—Ca(OH)₂, while low concentration of calcium oxide results in hydroxyapatite [Rodriguez-Lugo, V.; et al. Synthesis and structural characterization of hydroxyapatite obtained from CaO and CaHPO₄ by a hydrothermal method. Mater. Res. Innov. 2005, 9, 20-22]. Biphasic calcium phosphate (BCP) is an intimate mixture of two phases of HA and β-TCP (Ca₃(PO₄)₂) in variety of ratios, which appears after annealing of HA above 700° C. [ Kuo, M. C.; Yen, S. K. The process of electrochemical deposited hydroxyapatite coatings on biomedical titanium at room temperature. Mater. Sci. Eng. C 2002, 20, 153-160.].

Nanoslabs (graphene-like) of MoS₂ and numerous other layered materials are currently studied intensively for variety of optoelectronic as well as for energy harvesting and energy-storage devices [Manzeli, S.; et al. 2D transition metal dichalcogenides. Nat. Rev. Mater. 2 2017, 44, 16399-16404]. WS₂ and MoS₂ nanoparticles with fullerene-like (IF) structure were found to perform well as solid lubricants [Rapoport, L.; et al. Hollow nanoparticles of WS₂ as potential solid-state lubricants. Nature 1997, 387, 791-793; (ii) Rosentsveig, R.; et al.. Fullerene-like MoS₂ nanoparticles and their tribological behavior. Tribol. Lett. 2009, 36, 175-182]. They are presently used in various commercial products, mostly as additives to lubricating fluids, greases, metal working fluids and in high performance bearings.

Recently, doping of IF-MoS₂ nanoparticles with minute amounts (<200 ppm) of rhenium atoms (Re:IF-MoS₂) was demonstrated [ Yadgarov, L.; et al. Tribological studies of rhenium doped fullerene-like MoS₂ nanoparticles in boundary, mixed and elasto-hydrodynamic lubrication conditions. Wear 2013, 297, 1103-1110].

One of the most critical aspects of the usage of nanomaterials is their toxicity and biocompatibility. In contrast to various other nanoparticles, the IF NP were found to be non-toxic in general, up to a very high dosage (>100 μg/mL). These findings are beneficial for the development of medical technologies based on such nanoparticles.

In the present invention, HA based films are impregnated with doped inorganic fullerene-like (IF) nanoparticles (such as: Re:IF-WS₂, Nb:IF-WS₂, Re:IF-MoS₂, Nb:IF-MoS₂) or with doped inorganic nanotubes (INT) (such as: Re:INT-WS₂, Nb: NT-WS₂, Re:INT-MoS₂, Nb:INT-MoS₂) leading to substantial improvement in their tribological behavior.

### SUMMARY OF THE INVENTION

In some embodiments, this invention is directed to a composition comprising hydroxyapatite [Ca₁₀(PO₄)₆(OH)₂)] and inorganic fullerene-like (IF) nanoparticles or inorganic nanotubes (INT) doped by rhenium or niobium.

In some embodiments, this invention is directed to a film on a biocompatible substrate, wherein the film comprises hydroxyapatite [Ca₁₀(PO₄)₆(OH)₂)] and inorganic fullerene-like (IF) nanoparticles or inorganic nanotubes (INT) doped by rhenium or niobium.

In some embodiments, this invention is directed to a method of coating a biocompatible substrate or a metal substrate with the composition or the film of this invention, wherein the method comprises electrophoretic deposition plasma spray, ion beam coating, e-beam evaporation, thermal deposition, physical vapor deposition (PVD), aerosol deposition, vacuum deposition, sol gel deposition, or dip coating.

In some embodiments, this invention is directed to a dental or orthopedic implant comprising the composition and/or film of this invention.

In some embodiments, this invention is directed to a dental or orthopedic implant comprising a biocompatible substrate coated by the film of this invention.

In some embodiments, this invention is directed to a bone regeneration therapy comprising administering an artificial bone implant comprising the composition of this invention.

In some embodiments, this invention provides a method of osseointegration comprising contacting an artificial bone implant comprising the composition of this invention with a bone needs to be improved.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1C present SEM images of Re:IF-MoS₂ nanoparticles. FIG. 1A: shows high-resolution scanning electron microscope (HRSEM) micrograph of the Re-doped IF NP powder in In-lens detector 5kV. The oblate shape of the nanoparticles with smooth surfaces is clearly delineated. The size range of the nanoparticles is 70-170 nm with a minor content (<10%) of NP larger than 200 nm. FIG. 1B shows high-resolution transmission electron microscopy (HRTEM) image of one such nanoparticle made of some 20 closed and nested layers of MoS₂. The crystalline perfection and atomically smooth (sulfur-terminated) surface of the IF NP contributes to their excellent mechanical and tribological performance. FIG. 1C shows SEM view of an agglomerate of Re:IF-MoS₂ nanoparticles. The synthesized nanoparticles are highly agglomerated and must be deagglomerated before use. Light sonication suffices to disperse them well in aqueous or ethanolic suspensions.

FIGS. 2A-2B present HRSEM pictures of HA with Re:IF-MoS₂ nanoparticles coating obtained from solution A on porous titanium substrate in two magnifications: (FIG. 2A) 100 μm; (FIG. 2B) 2 μm. The film is continuous but visibly is heavily cracked.

FIGS. 3A-3D present HRSEM images of the HA film with Re:IF-MoS₂ obtained from solution A after 2 hours (FIG. 3A), 3 hours (FIG. 3B), and 4 hours (FIG. 3C) deposition. The Re:IF-MoS₂ nanoparticles in the film (FIG. 3C) are observed in the backscattering electron (BSE) mode (FIG. 3D). The arrows in FIG. 3D point on the Re:IF-MoS₂ nanoparticles occluded in the HA film.

FIGS. 4A-4B present zeta-potential vs. pH for Re:IF-MoS₂ nanoparticles. FIG. 4A presents zeta-potential vs pH for Re:IF-MoS₂ nanoparticles in solutions A, B and C. The (positive) zeta-potential of the solutions used for EPD of the HA+IF film are marked by enlarged symbols. FIG. 4B presents zeta-potential vs pH for Re:IF-MoS₂ nanoparticles in different solutions.

FIGS. 5A-5B present XRD patterns of the HA films incorporating Re:IF-MoS₂ nanoparticles. FIG. 5A presents the XRD patterns of the different coatings obtained from solution A (1), solution B (2) and solution C (3). FIG. 5B presents XRD pattern of the film obtained from solution A (3 h) after annealing (700° C. for 1 h). Here, a strong crystalline peak associated with calcium pyrophosphate phase (Ca₂(P₂O₇)) is observed. This phase is obtained through water evaporation from the HA (Ca₁₀(PO₄)₆(OH)₂) film. The presence of the Re:IF-MoS₂ nanoparticles did not change appreciably upon annealing, suggesting that these NPs are thermally stable at the annealing conditions.

FIG. 6 presents XRD patterns of films obtained from solution A without the Re:IF-MoS₂ NP (a) and (with the IF NP) for different deposition periods: after 2 hours (b), 3 hours (c) and 4 hours (d).

FIG. 7 presents Raman spectra of HA powder film without (a) and with the Re:IF-MoS₂ nanoparticles obtained from solution A for different EPD periods: after 2 hours (b), 3 hours (c) and 4 hours (d).

FIGS. 8A-8D present optical images of wear on the ball and inside the track of HA film without (FIG. 8A) and with the Re:IF-MoS₂ nanoparticles obtained from solution A for different periods: after 2 hours (FIG. 8B), 3 hours (FIG. 8C) and 4 hours (FIG. 8D) on anodized titanium.

FIGS. 9A-9B present SEM images of HA with Re:IF-MoS₂ nanoparticles coating obtained from solution B (3 h deposition time) on porous titanium substrate in different magnifications.

FIGS. 10A-10B present SEM pictures of HA with Re:IF-MoS₂ nanoparticles coating obtained from solution C (1 h deposition time) on porous titanium substrate in different magnifications

FIGS. 11A-11D SEM images of titanium surface (a,b) before and (c,d) after surface treatment in different magnifications.

FIGS. 12A-12C SEM images of porous titanium after anodization in different magnifications, the average diameter of the pores (tubes) is 100 nm.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In some embodiments, this invention is directed to a composition comprising hydroxyapatite [Ca₁₀(PO₄)₆(OH)₂)] and doped inorganic fullerene-like nanoparticles (IF-NPs) or doped inorganic nanotubes (INT).

In some embodiments, this invention is directed to a film comprising hydroxyapatite [Ca₁₀(PO₄)₆(OH)₂)] and doped inorganic fullerene-like nanoparticles (IF-NPs) or doped inorganic nanotubes (INTs).

In some embodiments, this invention is directed to a film comprising the composition of this invention.

In some embodiments, this invention is directed to a film comprising hydroxyapatite [Ca₁₀(PO₄)₆(OH)₂)] and doped inorganic fullerene-like nanoparticles (IF-NPs) or doped inorganic nanotubes (INT), wherein the film is coated on a solid substrate.

In other embodiments, the inorganic fullerene-like nanoparticles (IF-NPs) or the inorganic nanotubes (INT) are doped by rhenium and niobium.

Inorganic Fullerene-like (IF) nanoparticles and/or inorganic nanotubes (INT) of this invention each having the formula A₁₋ₓBₓ-chalcognide wherein A is a metal or transition metal or an alloy of one metals or transition metals including at least one of the following: Mo, W, Re, Ti, Zr, Hf, Nb, Ta, Pt, Ru, Rh, In, Ga, InS, InSe, GaS, GaSe, WMo, TiW; and B (dopant) is a metal transition metal selected from the following: Si, Nb, Ta, W, Mo, Sc, Y, La, Hf, Ir, Mn, Ru, Re, Os, V, Au, Rh, Pd, Cr, Co, Fe, Ni; and x is below or equal 0.003, and the chalcogenide is selected from the S, Se, Te. In other embodiments, x is below or equal 0.001.

For example, doped IF-NP or doped INT of the invention may be IF-Mo₁₋ₓNbₓS₂, IF-Mo₁₋ₓReₓS₂, INT-Mo₁₋ₓNbₓS₂, INT-Mo₁₋ₓReₓS₂, IF-W₁₋ₓNbₓS₂, IF-W₁₋ₓReₓS₂, INT-W₁₋ₓNbₓS₂, INT-W₁₋ₓRexS₂ or the alloys of WMoS₂, WMoSe₂, TiWS₂, TiWSe₂, where Nb or Re are doped therein. Within the alloys of the invention, taking WMo, TiW for example, the ratio between W and Mo or Ti and W may be 0.65-0.75 of one metal or transition metal and 0.25-0.35 of the other metal or transition metal, e.g. W_{0.7}Mo_{0.29}Nb_{0.01}S₂ (given with the percentage of the Nb dopant).

In one embodiment, the rhenium or niobium atoms serve as dopants in the lattice of the IF-NPs/INTs. The dopants substitute for the molybdenum or tungsten atoms, which lead to an excess of negative charge carriers being trapped on the IF-NPs/INT surfaces.

In other embodiments, the concentration of the dopants is below or equal to 0.3 at %. In other embodiments, the concentration of the dopants is between 0.01 to 0.1 at %. In other embodiments, the concentration of the dopants is between 0.01 to 0.07 at %. In other embodiments, the concentration of the dopants is between 0.01 to 0.05 at %.

The doped IF-nanoparticles/inorganic nanotubes behave like charged colloids, which do not agglomerate and form stable suspensions in oils and various fluids. This is in contrast to the undoped IF-NPs/INTs, as their structure allows rolling. Additionally, the doped IF-NPs and doped INTs have higher conductivity, higher carrier density, lower activation energy, and lower resistance than the undoped ones.

In some embodiments, this invention is directed to a composition or a film comprising hydroxyapatite [Ca₁₀(PO₄)₆(OH)₂), HA] and doped IF-NPs/doped INTs. In other embodiment, the composition of the film further comprises brushite, portlandite, other HA minerals or combination thereof.

In some embodiments, this invention is directed to a composition and/or a film comprising hydroxyapatite [Ca₁₀(PO₄)₆(OH)₂), HA] and doped IF-NPs or doped INTs. In other embodiment, the concentration of the doped IF-NPs or doped INTs is between 0.2 wt % to 5 wt % of the composition and/or film. In other embodiment, the concentration of the doped IF-NPs or doped INTs is between 0.2 wt % to 2 wt % of the composition and/or film. In other embodiment, the concentration of the doped IF-NPs or doped INTs is between 0.2 wt % to 1 wt %. In other embodiment, the concentration of doped IF-NPs or doped INTs is between 0.2 wt % to 1.5 wt %. In other embodiment, the concentration of the doped IF-NPs or doped INTs is between 0.5 wt % to 1.5 wt %. In other embodiment, the concentration of the doped IF-NPs or doped INTs is between 0.5 wt % to 2 wt %. In other embodiment, the concentration of the doped IF-NPs or doped INTs is between 1 wt % to 5 wt %. In other embodiment, the concentration of the doped IF-NPs or doped INTs is between 0.5 wt % to 3 wt %. In other embodiment, the concentration of the doped IF-NPs or doped INTs is between 1.5 wt % to 5 wt %.

In some embodiment, the film of this invention is coated on a solid substrate. In other embodiment, the solid substrate is biocompatible. In other embodiments, the solid substrate is metallic biocompatible. In other embodiments the solid and biocompatible substrate is titanium, alloys of titanium, Ti₆Al₄V, Co—Cr alloys, magnesium, stainless steel, shape memory alloys of nickel-titanium, silver, tantalum, zirconium, novel ceramics such as alumina or zirconia or any other electrical-conductive substrate.

In other embodiment, the titanium is porous.

In other embodiment, to improve the coating of the film onto the solid substrate the composition and/or film further comprises a cationic surfactant. In other embodiment a cationic surfactant comprises an ammonium group. Non limiting examples of cationic surfactant include: alkyltrimethylammonium salts: cetyl trimethylammonium bromide (CTAB) and cetyl trimethylammonium chloride (CTAC); benzalkonium chloride (BAC); cetylpyridinium chloride (CPC) or benzethonium chloride (BZT).

In other embodiment, to improve the coating of the film to the solid substrate the composition and/or film further comprises a polymeric binder. In other embodiments a non-limiting example of a polymer binder include a poly(lactic acid) (PLAs) based polymer.

In some embodiments this invention provides methods for coating a solid substrate with the composition of this invention to form a film on a solid substrate. In other embodiments, the methods of coating include; (i) electrophoretic deposition (solution); (ii) plasma spray (in vacuum); (iii) ion beam coating (in vacuum); (iv) e-beam evaporation [Cen Chen et al. Biomimetic apatite formation on calcium phosphate-coated titanium in Dulbecco's phosphate-buffered saline solution containing CaCl₂ with and without fibronectin, Acta Biomaterialia, (2010) 6, 2274-2281]; (v) thermal deposition; vacuum deposition [D. Predoi et al. Characteristics of hydroxyapatite thin films, J. Optoelect and Adv.Mat., (2007), 9(12), 3827-3831]; (vi) physical vapor deposition (PVD) [Ohad Goldbart et al.New Deposition Technique for Metal Films Containing Inorganic Fullerene-Like (IF) Nanoparticles, Chem Phys Chem, (2013), 14, 2125-2131; Olga Elianov MSc thesis submitted to the Faculty of Dental Medicine, Hadassah-Hebrew University, Jerusalem 91120, Israel (March 2018); (vii) aerosol deposition [C. Piccirillo, et al. Aerosol assisted chemical vapour deposition of hydroxyapatite-embedded titanium dioxide composite thin films, J. of photochem. And photobiol. A: Chemistry (2017), 332, 45-53]; (viii) sol gel deposition or (ix) dip coating. Each represents a separate embodiment of this invention.

### Electrophoretic Deposition

The electrophoresis coating technique is an inexpensive process capable of a high deposition rate while maintaining control of the coating thickness and morphology on the metal. In addition, this technique has a wide range of materials permitting coating of variety of shapes and sizes, all resulting in a quality surface with uniform thickness. The electrophoresis coating technique also has high material efficiency and can perform at low temperatures. The electrophoresis coating technique requires several steps, including surface treatments, which are used to clean the electrode from contaminants, improve the mechanical properties to create a uniform coating, and achieve better adhesion deposition. Electrophoresis coating is performed by dipping two electrodes into a container of electrolyte solution. A constant power supply creates an electrical field in the solution, which moves the charging colloid toward the opposite electrode. The deposition is obtained by chemical oxidation and reduction. The final step is an annealing process, and is done to achieve a smooth and continuous coating characterized by good adhesion to the surface. The electrophoretic deposition for coating the composition of this invention on a porous solid substrate is conducted at a relatively low temperature using an aqueous electrolyte containing calcium and phosphate salts. In this method, the calcium phosphate is deposited on the cathode as a result of a pH increase in the vicinity of the cathode and by the reduction of the H⁺ ion accompanying the generation of H₂ gas and OH. ions. The production of H₂ on the cathode's surface inhibits the nucleation or absorption of calcium phosphate on the cathode. Adding an alcohol such as ethanol to the electrolyte solution resolves this problem.

In some embodiment, this invention provides a method of coating a metal substrate with the composition of this invention, wherein the method comprises electrophoretic deposition having an electrolyte comprising a calcium salt, a phosphate salt and doped inorganic fullerene like nanoparticles, and thereby forming a film of desirable composition on the substrate.

The coating process of the film of this invention depends on achieving the proper pH solution that allows quality coatings, which in turn, relies on the nanoparticles' zeta potential measurement. In other embodiment, the composition has a positive zeta potential at pH below 6.5. In neutral pH (7) the nanoparticles are negatively charged, which reflects the extra negative charge induced by native defects in the lattice and chemisorbed negatively charged moieties, like OH— groups. This extra negative charge is neutralized in very low pH (up to pH=2) by positively charged chemical moieties, like protons, etc. In either very low and very high pH, the Debye screening radius is very small (few nm) leading to agglomeration of the nanoparticles and their precipitation. Thus, the electrophoresis coating process of the composition of this invention is performed at pH 6-7 to: 1) avoid damaging the surface of the nanoparticles; 2) provide a stable working solution; and 3) achieve a uniform coating of the substrate. Within this pH range, the nanoparticles gained a negative charge and the deposition was performed on the anode.

In some embodiments the methods for coating a metal substrate with the composition of this invention is performed by electrophoretic deposition. In another embodiment, the metal substrate is pretreated for example with carbon paper to obtain a smooth surface and then the metal substrate is anodized prior to the electrophoretic deposition. In other embodiment, the metal substrate is anodized in electrolyte solution containing a fluoride ion. In other embodiment, the elecrophoretic deposition is conducted as presented in Example 1.

Anodization is an electrochemical method for producing a protective layer on metal by forming a metal oxide layer which makes the metal substrate biocompatible. The metal oxide layer is a few tens of microns thick with micro pores to maintain homogeneity. The anodization process creates a porous surface, which improves and increases osseointegration (the functional connection between the human bone and the implant), and thereby increase the osteoblast adhesion (bone cell).

In another embodiment the electrophoretic deposition (EPD) is conducted between 2 to 5 hours. In another embodiment the electrophoretic deposition is conducted for 2, 3, 4 or 5 hours. Each represents a separate embodiment of this invention.

In some embodiments, this invention provides HA coatings containing up to 5 wt % doped IF-NPs or doped INTs deposited on a porous metallic biocompatible substrate by electrophoretic deposition using DC bias. The major phase in each coating is hydroxyapatite which incorporates small amounts of doped IF-NPs or doped INTs. In other embodiments, the metal substrate was a titanium substrate. In other embodiments, the doped inorganic fullerene-like nanoparticle is Re:IF-MoS₂. In other embodiments, the doped inorganic fullerene-like nanoparticle is Re:IF-WS₂. In other embodiments, the doped inorganic fullerene-like nanoparticle is Nb:IF-MoS₂. In other embodiments, the doped inorganic fullerene-like nanoparticle is Nb:IF-WS₂. In other embodiments, the doped inorganic nanotube is Re:INT-MoS₂. In other embodiments, the doped inorganic nanotube is Re:INT-WS₂. In other embodiments, the doped inorganic nanotube is Nb:INT-MoS₂. In other embodiments, the doped inorganic nanotube is Nb:INT-WS₂.

In some embodiments, the film formed on the metal substrate has low friction coefficient of between 0.05 to 0.15. In another embodiment, the film formed by EPD on the metal substrate has low friction coefficient of between 0.05 to 0.1. In another embodiment, the low friction is maintained after annealing. In another embodiment, the film maintains its mechanical robustness.

### Uses Thereof

Artificial bone implants became a major health concern. Hydroxyapatite (Ca₁₀(PO₄)₆(OH)₂; HA) is the main constituency of the bone. Hydroxyapatite, is chemically similar to the calcium phosphate mineral present in bone and biological hard tissue.

The composition and film of this invention are for use in dental and orthopedic implants having very low friction, good adhesion to the underlying rough substrate even under very high load (600 MPa). The composition and film of this invention have high biocompatibility, specifically as a bone substitute. The composition/film prepared by the methods of this invention form a homogeneous structure, having slow degradability rate and both osteointegration and osteoconductive characteristics, which improve bone growth.

In some embodiments, this invention provides a dental or orthopedic implant comprising the composition of this invention. In other embodiments, this invention provides a dental or orthopedic implant comprising a film on a biocompatible substrate, wherein the film comprises the composition of this invention.

In some embodiments, this invention provides a bone regeneration therapy comprising administering an artificial bone implant comprising the composition of this invention.

In some embodiments, this invention provides a method of osseointegration comprising contacting an artificial bone implant comprising the composition of this invention in a bone needs to be improved. In other embodiments, the artificial bone implant comprises a biocompatible substrate coated by a film, wherein the film comprises the composition of this invention.

The methods of this invention for osseointegration or for bone regeneration provide fast fixation and spontaneous binding of the HA to neighboring bone, having osteoconductive properties, resulting in deposition of biological apatite on the surface of the implant and thereby bone healing around the implant.

The following examples are presented in order to more fully illustrate the preferred embodiments of the invention. They should in no way be construed, however, as limiting the broad scope of the invention.

### EXAMPLES

### Example 1

### Preparation of a Film of Hydroxyapatite (HA) and Rhenium Doped Fullerene Like MoS₂ (Re:IF-MoS₂) on Titanium Substrate

A titanium electrode (30×5×0.3 mm, 97 wt % purity) was polished with silicon carbide paper to a mirror finish. It was subsequently cleaned by sonicating in a series of solvents, i.e., acetone, ethanol, methanol, isopropanol and finally distilled water, then dried under a nitrogen stream.

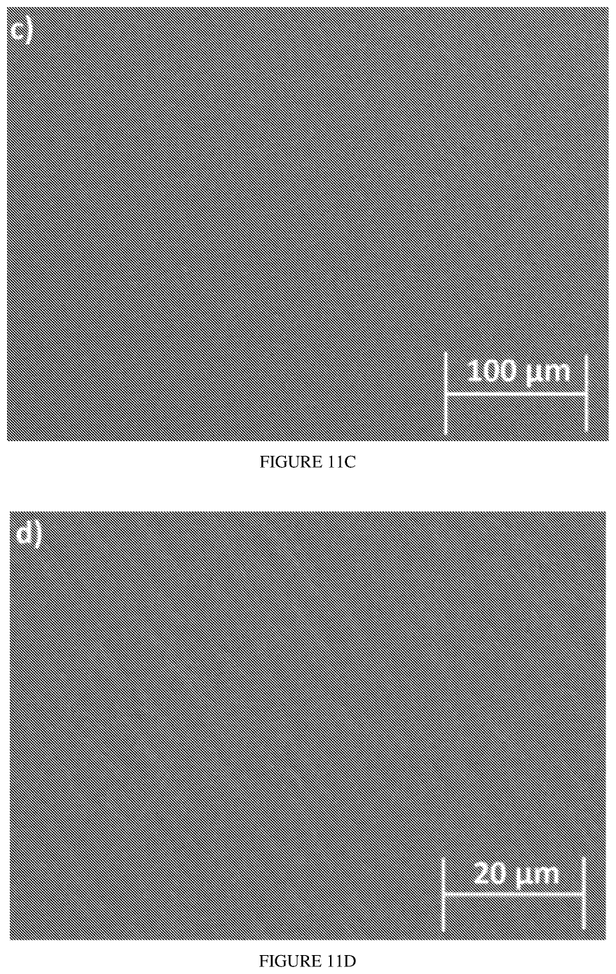

The surface morphology of the titanium before the pretreatment preceding the anodization is presented in FIGS. 11A-11B. Visibly, the fresh surface was heavily contaminated with a dense network of scratches. After treatment of the titanium with different solvents, a smooth surface with low density of scratches and clean from contaminants was obtained (FIGS. 11C-11D). The smooth surface was imperative for achieving reproducible tribological measurements.

### Titanium Anodization

An electrochemical cell containing two-electrodes, i.e., platinum (cathode) and titanium (anode) was used. The electrolyte solution contained 1 M (NH₄)₂SO₄ and 0.5 wt % NH₄F. All electrolytes were prepared from reagent grade chemicals and deionized water. The electrochemical treatment was conducted with a DC power source operated at 2.5 V and 1.5 A, at room temperature for 2.5 h. After the electrochemical treatment, the samples were rinsed with deionized water and dried under nitrogen stream.

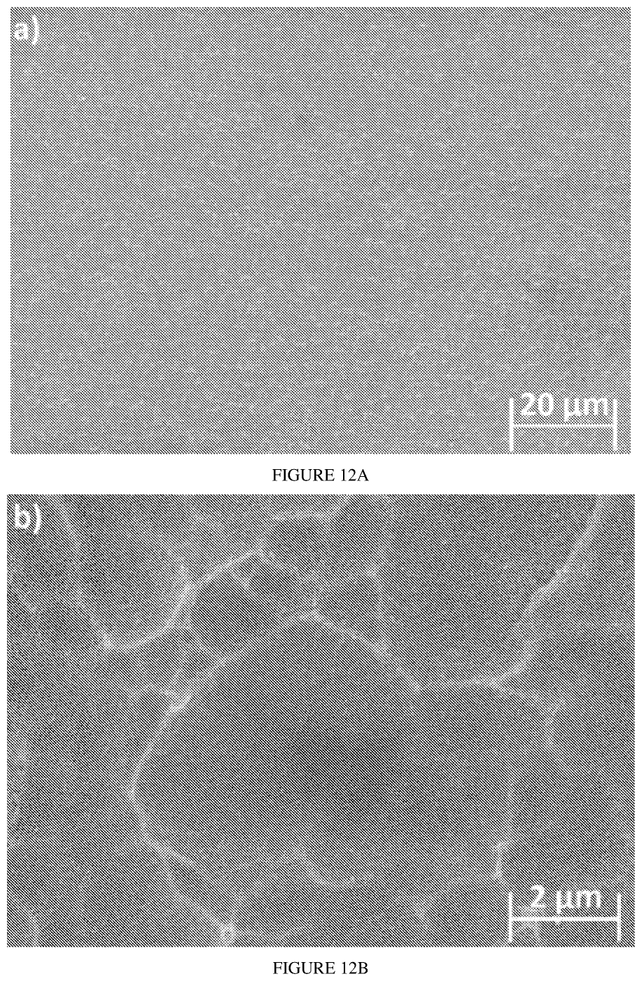

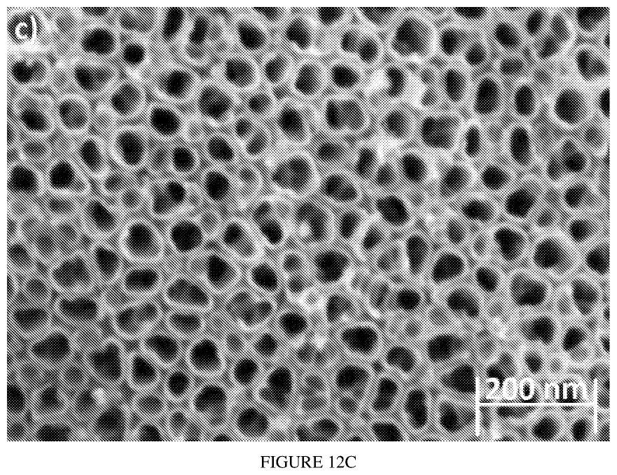

The surface of the titanium after anodization is displayed in FIGS. 12A-12C. Visibly the anodized titanium surface consists of a dense array of (TiO₂) nanotubes with the range of pore diameters between 50-130 nm, which form a highly organized, roughly hexagonal, pattern on the Ti surface.

### Electrophoretic Deposition (EPD)

The detailed synthesis of the Re:IF-MoS₂ nanoparticles (Re content <0.1 at %), which were added to the coating processes, was reported before [Yadgarov, L.; et al. Investigation of Rhenium-Doped MoS₂ Nanoparticles with Fullerene-Like Structure. Z. Anorg. Allg. Chem. 2012, 638, 2610-2616]. Three different chemical baths were used for electrophoretic deposition of HA+IF NP on the porous titanium substrate. Titanium samples were used as the working electrode (cathode), while a platinum plate served as the anode. The final volume of all three electrolyte solutions containing 1 mg of the IF NP was 50 mL.

Solution A: The electrolyte solution consisted of 42 mM Ca(NO₃)₂ and 25 mM NH₄H₂PO₄, 1 mg Re:IF-MoS₂ sonicated in 3 mM cetyl trimethylammonium bromide (CTAB). Ethyl alcohol was added into the above solution in a 1:1 ratio in order to reduce the hydrogen evolution on the titanium electrode. The initial pH of the electrolyte solution was 4.5. The coating process was carried out at 40° C. with a DC power supply at 20 V bias and 0.11 A for 3 h. The samples were washed with deionized water and dried for 24 h at 100° C.

Solution B: The electrolyte solution consisted of 5.25 mM Ca(NO₃)₂, 10.5 mM NH₄H₂PO₄, and 150 mM NaCl. The initial pH of the solution was adjusted to 5.30 by adding NaOH. 1 mg Re:IF-MoS₂ was sonicated in distilled water for 15 min and added to the electrolyte solution. The coating process was conducted with a DC power source operated at 2.5 V and 0.11 A at room temperature for 3 h.

Solution C: The electrolyte solution consisted of 3 mM Ca(NO₃)₂ and 1.8 mM KH₂PO₄, 1 mg Re:IF-MoS₂ sonicated in 3 mM CTAB. The initial pH of the electrolyte solution was 5. The coating process was conducted with a DC power source operated at 6 V and 1 A at room temperature for 1 h. The resulting samples, after coating, were washed with deionized water and dried in room temperature.

The formal molar Ca/P ratio in HA is 5:3 (1.67). The Ca/P ratio in each coating was calculated based on semi-quantitative. Energy dispersive spectroscopy (EDS) analysis. For solution A, the ratio was found to be 2.6. The higher abundance of calcium in this coating could be attributed to the presence of portlandite (Ca(OH)₂). The Ca/P ratio of the coating obtained from solution B, which was highly crystalline and discontinuous was 1.5, which agrees well with the HA formula (1.66). The ratio is 1 for the coating obtained from solution C, which can be ascribed to the presence of calcium pyrophosphate phase (Ca₂(P₂O₇)) in the coating—see XRD analysis (Example 3).

The bath showing the most uniform coating and good adhesion (solution A) was then further studied by changing the deposition time to 2, 3 and 4 hours and subsequent annealing at 700° C. for 1 h.

### Characterization

### High-Resolution Scanning Electron Microscopy (HRSEM) and High-Resolution Transmission Electron Microcopy (HRTEM)

The surface morphology of the titanium samples was analyzed by (HRSEM) (Zeiss Ultra 55) after each step. For topographical information, the secondary electrons were recorded using the SE2 and In-lens detectors. For atomic number contrast the backscattering electron (BSE) detector was used. In order to avoid the sample charging during the analysis, the imaging was done under relatively low accelerating voltage (2-5 kV) and low current. Energy dispersive spectroscopy (EDS) analysis (EDS Bruker XFlash/60 mm) of the samples was undertaken as well. The reported results of the EDS were based on standard-less analysis and hence is semi-quantitative in nature.

TEM was performed with a JEOL 2100 microscope (JEOL Ltd., Tokyo, Japan) operating at 200 kV, equipped with a Thermo Fisher EDS analyzer. High-resolution TEM (HRTEM) images were recorded with a Tecnai F30 UT (FEI) microscope (FEI, Eindhoven, the Netherlands) operating a 300 kV. The TEM grids were prepared by dripping an ethanolic solution of the nanoparticles onto a collodion-coated Cu grids.

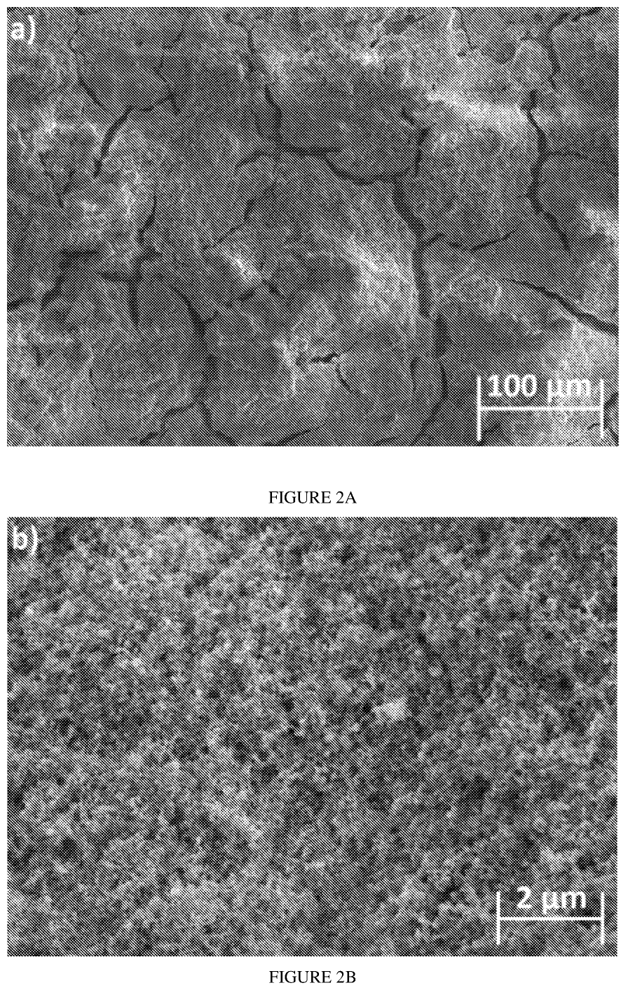

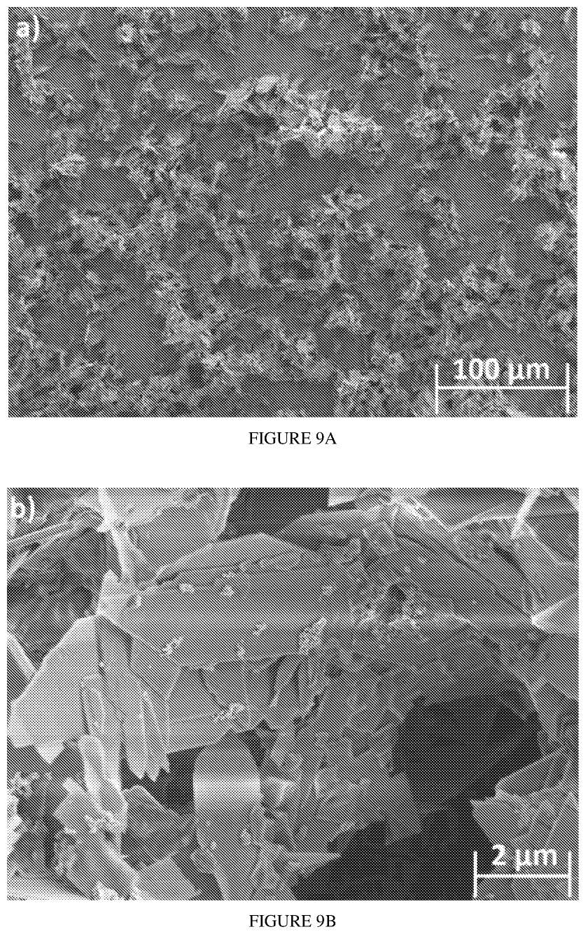

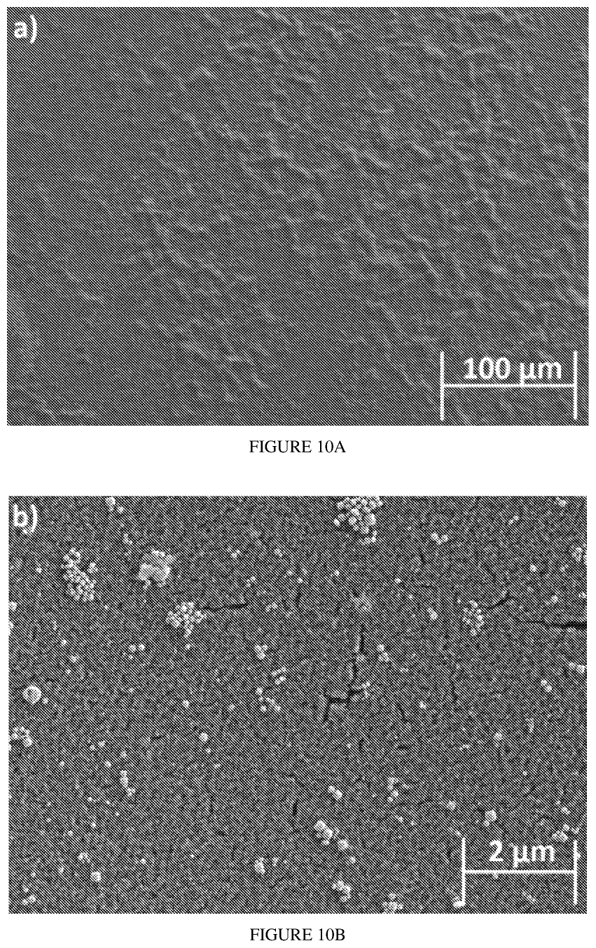

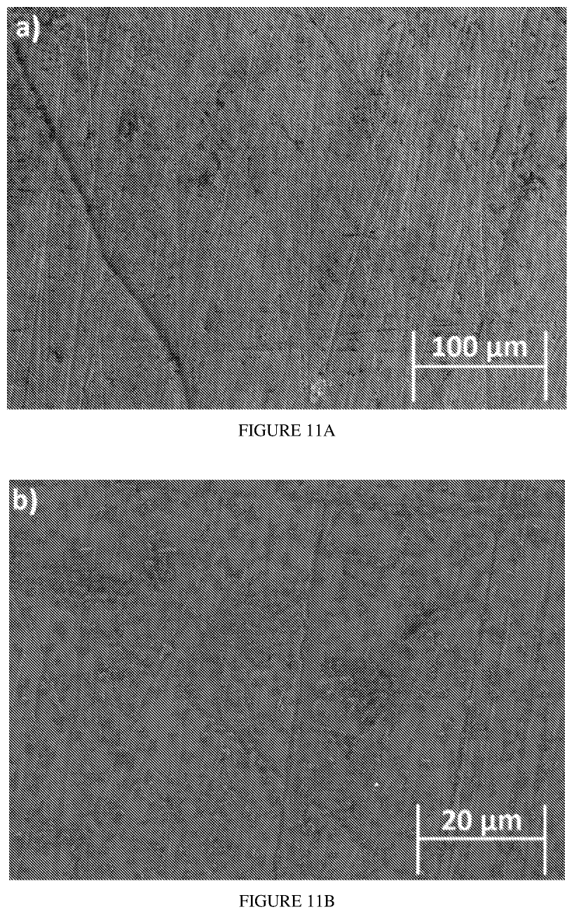

The surface morphology of the HA film prepared via solution A (FIGS. 2A-2B) and solution C was more homogeneous and could be successfully combined with the Re:IF-MoS₂ NP in the films as opposed to the film obtained from solution B, which was highly crystalline but non-uniform. The surface morphology of the film obtained from solutions B and C are shown in FIGS. 9A-9B and 10A-10B, respectively

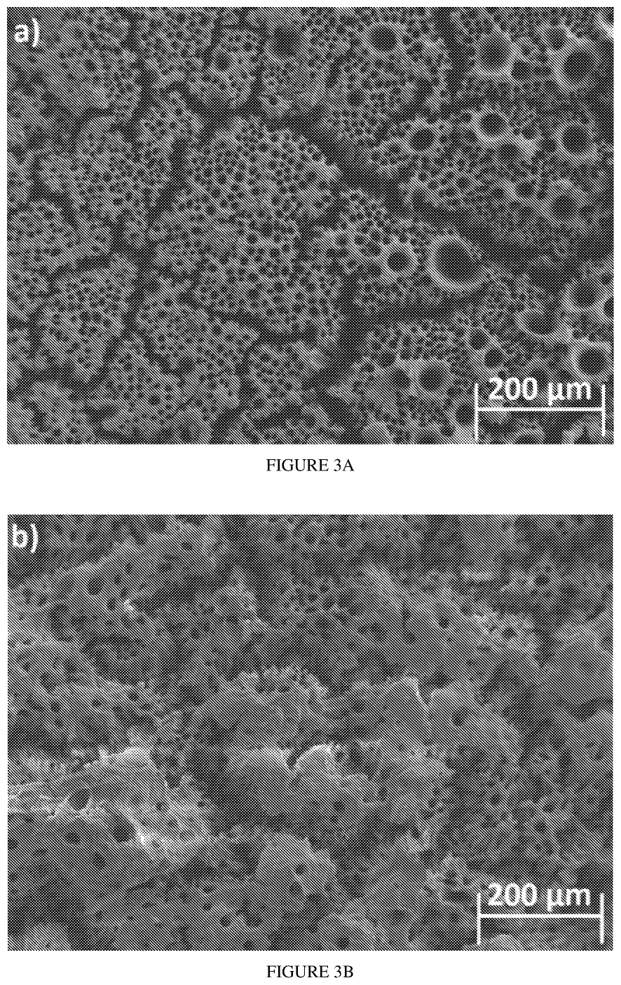

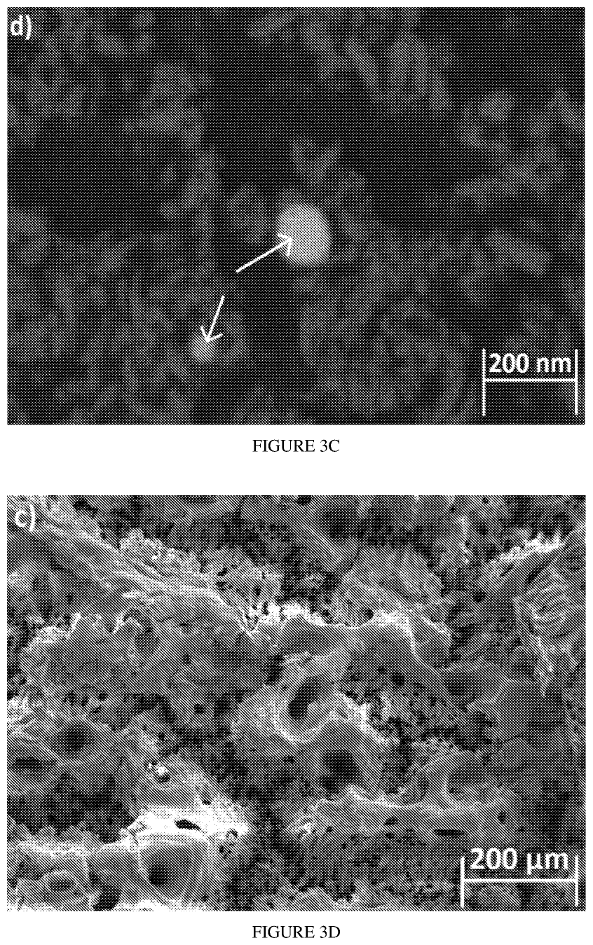

The SEM images of the surface of the HA films with Re:IF-MoS₂ nanoparticles obtained from solution A for different deposition periods are shown in FIGS. 3A-3D. The surface of the coated film shows defects, including the presence of cracks and pores with circular shape. Such pores can be probably attributed to the formation of H₂(g) bubbles during the coating process.

Interestingly, the bias applied during EPD for solution B (and C) was appreciably smaller (2.5 V) compared to solution A (20 V). On the other hand, the film obtained by EPD from solution A was quasi-continuous. It was highly crystalline but less uniform in the case of solution B, i.e., the apparent current density was higher than that calculated on the basis of the formal electrode surface. The higher voltage used for the EPD from solution A implied a much higher rate of hydrogen production, which could explain the porous structure of this film. The density of the pores and their sizes could be possibly tuned by the bias applied on the cathode during the electrophoretic deposition. Furthermore, addition of surface active agents, like CTAB and others, could reduce the size of the pores.

The large cracks are diminished, and the pore-size decreased as the coating time was prolonged. The thickness of the coating was a few microns, therefore the nanoparticles could have been buried under the film surface and even be closer to the titanium substrate. Using low energy beam (2 keV) in the BSE mode, the IF NP could be nevertheless observed (FIG. 3D).

### Example 2

### Zeta Potential Results of Hydroxyapatite (HA) and Rhenium Doped Fullerene Like MoS₂ (Re:IF-MoS₂) Film on Titanium Substrate

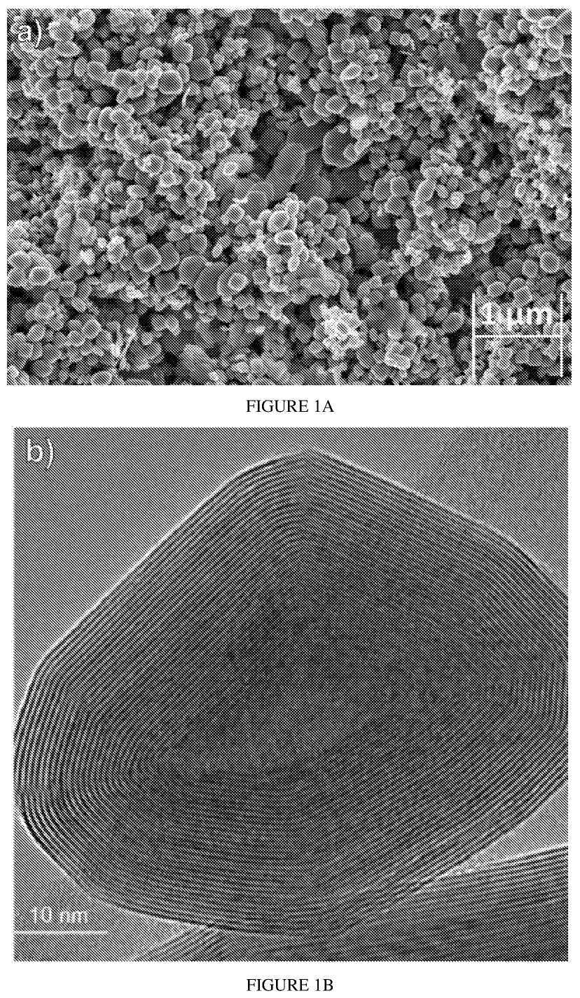

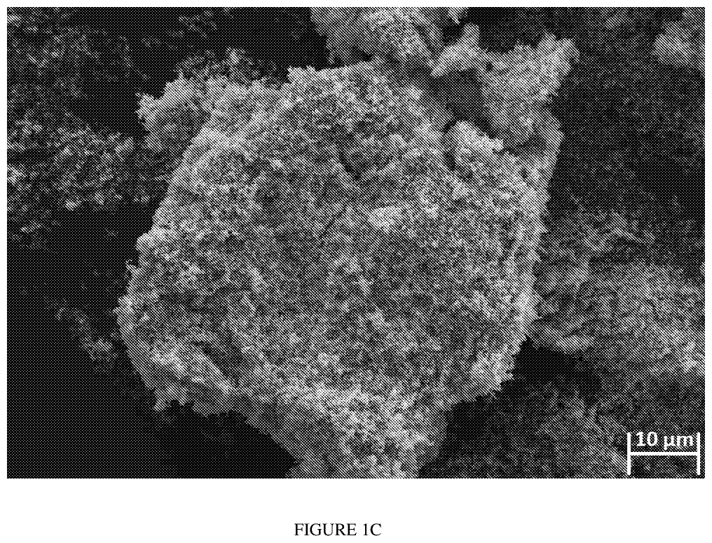

The surface charge of the HA suspension with and without the nanoparticles was determined by zeta potential (ZP) measurements using ZetaSizer Nano ZS (Malvern Instruments Inc., Malvern, UK) with a He—Ne light source (632 nm). To prepare the samples for these measurements, IF (0.6 mg) NP were deagglomerated in 20 mL purified water by sonicating for 5-10 minutes using an ultrasonic bath (see FIG. 1C for a SEM image of such an agglomerate). Subsequently, 0.2 mL of the IF suspension was added to 1.5 mL aqueous solutions with pH varying from 1 to 12 and sonicated for an extra 5 min. Before the addition of the IF NP, the pH of each solution was adjusted using concentrated NaOH or HCl. The final concentration of the IF NP was 0.004 mg/mL. The ZP of the solutions was measured in a folded capillary cell (DTS1060) made from polycarbonate with gold plated beryllium/copper electrodes.

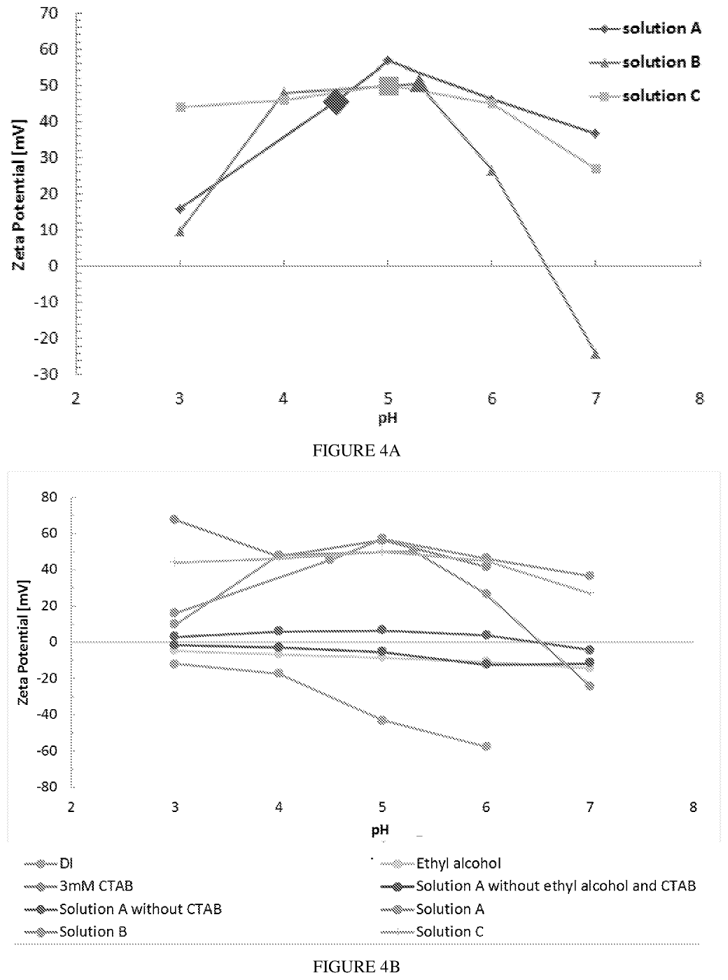

FIGS. 4A-4B show the results of the Zeta potential (ZP) measurements performed with the three solutions containing Re:IF-MoS₂ nanoparticles as a function of pH—up to pH=7. The ZP of all the solutions containing the nanoparticles was positive for pH below 6.5. At higher pH the ZP of solution B became negative, while that of solutions A and C remain positive. This difference can be attributed to the addition of the CTAB, which is a cationic surfactant, to solutions A and C. The (positive) ZP of the natural solutions used for EPD is marked on FIG. 4A for all three solutions.

The ZP measurements showed that the species in the HA solution containing the IF NP were positively charged and consequently, the HA film could be deposited on the negative electrode (Ti) during the EPD process. The ZP of the IF NP in pure water, ethanol solution, CTAB in water, and the three solutions used for the EPD (included also in FIG. 4A) are summarized in FIG. 4B, the errors of the ZP measurements were about 2%.

### Example 3

### X-Ray Diffraction (XRD) of Hydroxyapatite (HA) and Rhenium Doped Fullerene Like MoS₂ (Re:IF-MoS₂) Film

The film was removed from the Ti substrate and carefully crushed into a powder. The powder was analyzed by X-ray powder diffraction (XRD) using TTRAX III (Rigaku, Tokyo, Japan) theta-theta diffractometer equipped with a rotating copper anode X-ray tube operating at 50 kV/200 mA. A scintillation detector aligned at the diffracted beam was used after a bent Graphite monochromator. The samples were scanned in specular diffraction mode (θ/2θ scans) from 10 to 80 degrees (2θ) with step size of 0.025 degrees and scan rate of 0.5 degree per minute. Phase identification and quantitative analysis were performed using the Jade 2010 software (MDI) and PDF-4+ (2016) database.

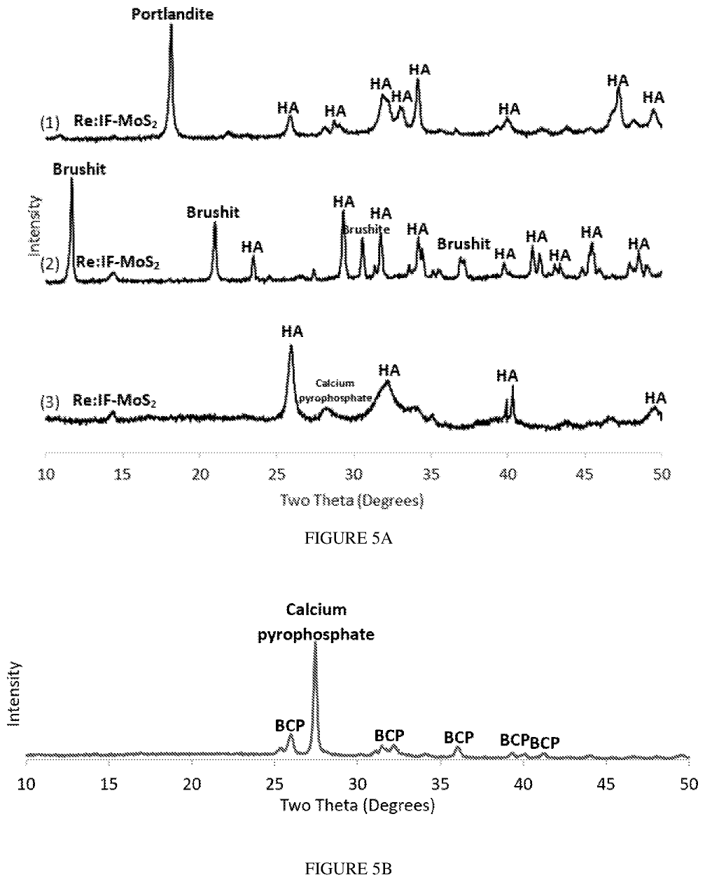

The results of the XRD analyses are summarized in FIGS. 5A-5B and in Table 1. The XRD patterns of the different coatings obtained from solutions A, B and C are shown in FIG. 5A. The major phase obtained by EPD of these solutions is HA. Nonetheless, the coating obtained from solution A contained appreciable amounts (25 wt %) of portlandite (Ca(OH)₂). Solution B, on the other hand, contained, in addition to the HA, also significant amounts of brushite—(CaH(PO₄)·2H₂O). The film obtained from solution C contained calcium pyrophosphate—(Ca₂(P₂O₇)). The presence of the Re:IF-MoS₂ nanoparticles in the coatings is confirmed by the tiny peak at 14.3°. The content of the IF NP is calculated as 0.2 wt % for solution A, 1.5 wt % for solution B and 1.4 wt % for solution C. This amount is rather small but could nevertheless lead to major improvements of the tribological properties of the film without compromising its mechanical robustness.

Following the annealing of the film obtained from solution A (FIG. 5B), the HA became biphasic calcium phosphate (BCP), i.e., intimate mixture of two phases: HA (73.6 wt %) and β-TCP (5.9 wt %), and 0.1 wt % Re:IF-MoS₂ NP.

TABLE 1
Composition of the films deposited from different
solutions determined from the XRD analysis.

| EPD | | | | Calcium | | Re:IF- |
|---|---|---|---|---|---|---|
| films | HA | Portlandite | Brushite | Pyrophosphate | β-TCP | MoS₂ |
| Film obtained | 74.8 wt % | 25 wt % | | | | 0.2 wt % |
| from solution A | | | | | | |
| Film obtained | 17.2 wt % | | 81.3 wt % | | | 1.5 wt % |
| from solution B | | | | | | |
| Film obtained | 81.1 wt % | | | 17.5 wt % | | 1.4 wt % |
| from solution C | | | | | | |
| Film obtained | 73.6 wt % | | | 20.4 wt % | 5.9 wt % | 0.1 wt % |
| from solution A | | | | | | |
| after annealing | | | | | | |

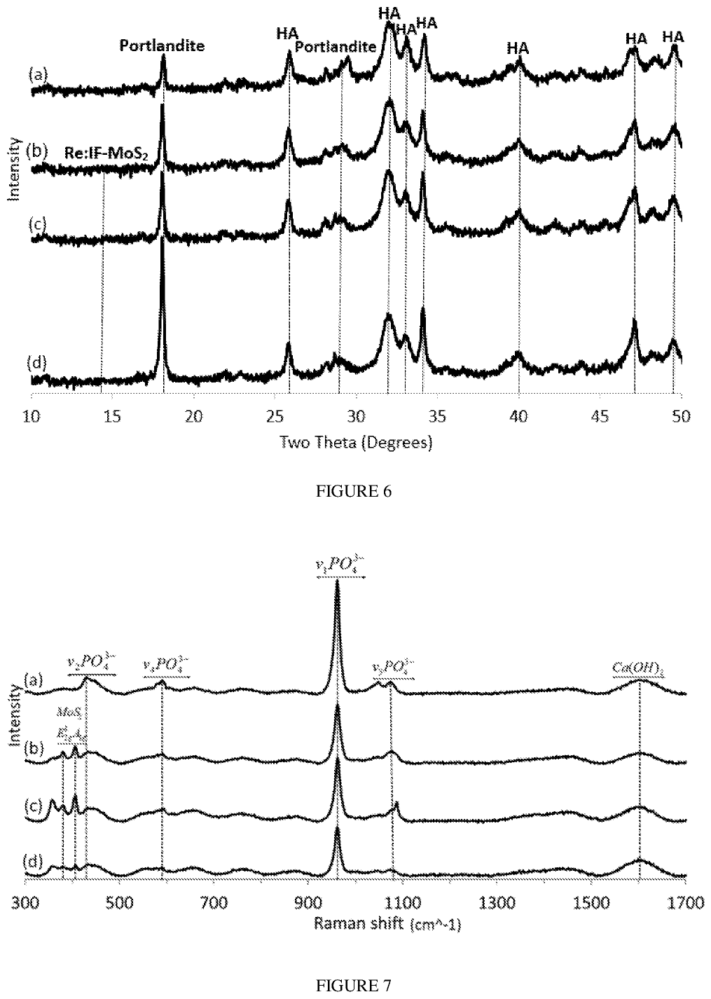

The XRD patterns of the films obtained from solution A without the NP (a) and with the IF NP for different deposition times (b-c) is shown in FIG. 6. The percentages of the compounds in each film is presented in Table 2. The major phase in the films was hydroxyapatite. The relative amount of the portlandite in the film increased with extending deposition times (FIG. 6). The relative amount of the calcium oxide didn't vary with the deposition time which was also true for the relative content of the IF NP. Although the signal of the IF NP was non-visible in FIG. 6, their presence is confirmed through both electron microscopy (FIGS. 3A-3D) and the Raman measurements (FIG. 7).

TABLE 2
Composition of the film determined via XRD analysis
for different deposition times (from solution A).

| | | | Calcium | |
|---|---|---|---|---|
| EPD films | HA | Portlandite | Oxide | Re:IF-MoS₂ |
| Film obtained | 87.8 wt % | 4.6 wt % | 7.6 wt % | |
| from solution | | | | |
| A without | | | | |
| Re:IF-MoS₂ (3 h) | | | | |
| Film obtained | 82.6 wt % | 7.4 wt % | 9.1 wt % | 0.3 wt % |
| from solution | | | | |
| A (2 h) | | | | |
| Film obtained | 80.4 wt % | 11.3 wt % | 8.0 wt % | 0.3 wt % |
| from solution | | | | |
| A (3 h) | | | | |
| Film obtained | 77.8 wt % | 13.6 wt % | 8.3 wt % | 0.3 wt % |
| from solution | | | | |
| A (4 h) | | | | |

### Example 4

### Raman Spectroscopy of Hydroxyapatite (HA) and Rhenium Doped Fullerene Like MoS₂ (Re:IF-MoS₂) Film

Raman spectra of the powders ground from the films were obtained with Horiba-Jobin Yivon (Lille, France) LabRAM HR Evolution set-up using solid state laser with a wavelength of 532 nm. The instrument was equipped with Olympus objectives MPlan N 100×NA 0.9. The measurements were conducted using a 600 grooves/mm grating. Each spectrum was acquired for 20 s and the spectra were averaged 100 times, which enabled using low excitation power thereby preserving the sample integrity. The spectral ranges collected were from 100 to 1800 cm⁻¹.

The Raman spectra of HA+IF films prepared from solution A for different deposition times (2, 3 and 4 hours) are shown in FIG. 7. The spectra showed the characteristic vibration bands of calcium hydroxide (wide peak at 1600 cm⁻¹) and poorly crystalline phosphoric moieties, especially phosphate PO₄⁻³ bands at 469 (ν₂), 562-603 (ν₄), 962 (ν₁) and 1000-1104 cm⁻¹ (ν₃). These bands are typical of HA. The Raman spectra showed also the typical MoS₂ modes at 383 (E_{2g}) and 408 cm⁻¹ (A_{1g}). Interestingly, in contrast to the XRD pattern (FIG. 6), the Raman bands of the IF NP in the HA film are easily discerned here.

### Example 5

### Tribological Results of Hydroxyapatite (HA) and Rhenium Doped Fullerene Like MoS₂ (Re:IF-MoS₂) Film

A home-made ball-on-flat rig was used for the tribological tests. The tests were carried-out at room temperature and humidity of ˜40%. Each test was repeated 5-times. Tribological tests were performed on the titanium samples at every step of the experimental procedure. The tribological testing was done under dry friction conditions. This testing method utilizes flat lower samples and a ball-shaped upper specimen, which slides against the flat specimen. The two surfaces move relative to each other in a linear, back and forth sliding motion, under a prescribed set of conditions. In this testing method, the load is applied vertically downwards through the ball against the horizontally mounted flat specimen. Two measurements procedures were used in these series of tests. Sliding speed of 0.3 mm/s was common to both series. In one series of measurements the load was 10 g; the diameter of the ball (hard steel—AISI 301) was 10 mm and consequently a Hertzian pressure of 150 MPa was applied on the film (20 cycles). In another series, the load was 20 g, the diameter of the ball 2 mm, i.e., a Hertzian pressure of 600 MPa was applied, and the number of cycles was 100.

Table 3 summarizes the data for the friction coefficient and surface roughness of the different samples under dry conditions. In general, the friction coefficient was found to go down along with the stages of the experimental procedure of preparing the film. The low friction coefficient of the HA film obtained from solution A can be attributed to the IF nanoparticle structure. The nanoparticles exhibited facile rolling when released from the film surface. In addition, gradual peeling/crushing of the NP and material transfer from the film surface to the counter surface of the ball contributed to the facile shearing of the mating surfaces and low friction coefficients. Interestingly, the friction coefficient of the HA film obtained from solution A was maintained also after 700° C. annealing.

TABLE 3
Summary of the initial and final friction coefficients and the initial roughness
for different stages of preparation of the composite HA + IF film.

| | Initial | Final Coefficient | Initial |
|---|---|---|---|
| | Coefficient | of Friction (after | Roughness |
| Tested film | of Friction | 20 Cycles) | (μm) |
| Titanium after surface treatment | 0.50 ± 0.01 | 0.60 ± 0.02 | 0.23 ± 0.03 |
| Titanium after anodization | 0.15 ± 0.01 | 0.23 ± 0.03 | 0.50 ± 0.05 |
| Film of HA with Re:IF-MoS₂ NP | 0.11 ± 0.01 | 0.13 ± 0.01 | 0.45 ± 0.4 |
| obtained from solution A on | | | |
| anodized titanium | | | |
| Film of HA with Re:IF-MoS₂ NP | 0.21 ± 0.02 | 0.43 ± 0.08 | 0.37 ± 0.03 |
| obtained from solution B on | | | |
| anodized titanium | | | |
| Film of HA with Re:IF-MoS₂ NP | 0.37 ± 0.23 | 0.30 ± 0.18 | 0.52 ± 0.02 |
| obtained from solution C on | | | |
| anodized titanium | | | |
| Film of HA with Re:IF-MoS₂ NP | 0.12 ± 0.01 | 0.11 ± 0.02 | 0.49 ± 0.7 |
| obtained from solution A on | | | |
| anodized titanium after annealing | | | |

Measurement conditions: diameter of the test ball 10 mm; load = 10 g (Hertzian pressure—P = 150 MPa).

Table 4 shows the dry friction coefficient of the coatings obtained from solutions A without (3 h) and with the NP after 2, 3 and 4 h of deposition time on the anodized titanium substrate. A higher Hertzian pressure (600 MPa) was used for the tribological test. The dry friction coefficient was reduced with increasing coating-time of the film.

Following the 4 h deposition time the friction coefficient was very low (0.12) attesting to the quality of the composite film.

TABLE 4
The initial and final friction coefficients and the initial
roughness of the coating on titanium substrate obtained
from solution A for different periods of deposition.

| | Initial | Final Coefficient | Initial |
|---|---|---|---|
| | Coefficient | of Friction (after | Roughness |
| Tested film | of Friction | 100 Cycles) | (μm) |
| Pure HA film | 0.66 ± 0.08 | 0.78 ± 0.04 | 1.59 ± 0.28 |
| obtained from | | | |
| solution A | | | |
| without NP after | | | |
| 3 h deposition | | | |
| HA film with | 0.75 ± 0.05 | 0.63 ± 0.03 | 0.49 ± 0.05 |
| Re:IF-MoS₂ NP | | | |
| obtained from | | | |
| solution A | | | |
| after 2 h | | | |
| HA film with | 0.53 ± 0.03 | 0.55 ± 0.04 | 0.57 ± 0.17 |
| Re:IF-MoS₂ NP | | | |
| obtained from | | | |
| solution A | | | |
| after 3 h | | | |
| HA film with | 0.13 ± 0.01 | 0.12 ± 0.02 | 0.48 ± 0.02 |
| Re:IF-MoS₂ NP | | | |
| obtained from | | | |
| solution A | | | |
| after 4 h | | | |

Measurement conditions: diameter of the test ball 2 mm; load 20 g and Hertzian pressure of P = 600 MPa.

Therefore, it is clear that the extended deposition of the composite film resulted in lower friction under very high load. However, the mechanical stability of the film might have been partially compromised. The surface roughness of the films was in the sub-micron range for all the films containing the NP.

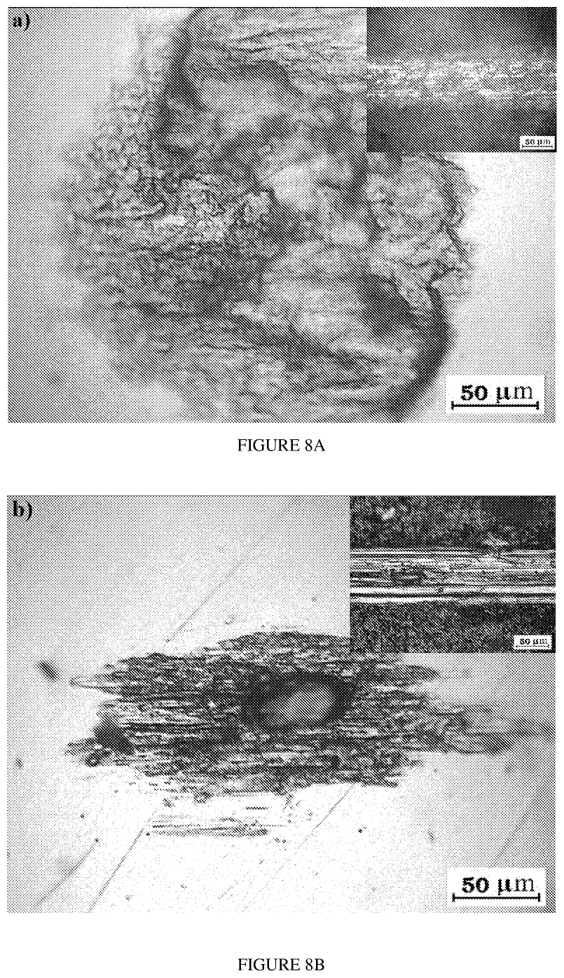

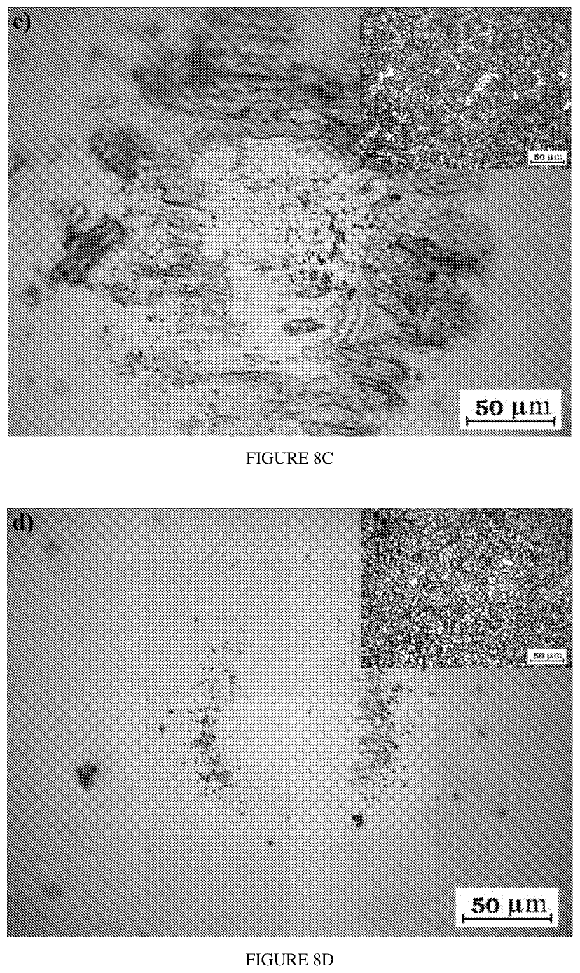

FIGS. 8A-8D show optical micrographs of the wear of the ball and the wear track on the film (inset) after different periods of EPD (600 MPa) and 100 cycles. In analogy to the friction coefficient, the visible wear scar on the ball and the wear track on the film were markedly reduced with the deposition time of the HA+IF NP film.

### Example 6

### Methods for Film Formation

### Sol-Gel Deposition

A solution of 3 M (C₂H₅O)₃PO was hydrolyzed for 24 h in a sealed container under vigorous stirring, 3 M Ca(NO₃)₂4H₂O was added dropwise with 1 mgr Re:IF-MoS₂ nanoparticles in anhydrous ethanol. The mixed sol solution agitated for additional 30 min and kept static at ambient duration time for 24 h. Ti₆Al₄V substrate was dip coated in the sol solution, then dried at 80° C. and followed by annealing in vacuum at 900° C. for 5 h.

### Dip Coating

3 mM Ca(NO₃)₂, 1.8 mM KH₂PO₄ were dissolved in distilled water, then adding 1 mgr Re:IF-MoS₂ nanoparticles after dispersion. Immersing titanium substrate in the solution at 37° C. and sealied the container for 24 h, finally the substrate was drying at room temperature, followed by annealing at 700° C. for 3 h.

### Casting Molding

PLLA was dissolved in dichloromethane and adding hydroxyapatite powder with Re:IF-MoS₂ nanoparticles to the polymer solution, split the solution to Teflon mold and drying at room temperature.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A composition comprising hydroxyapatite [Ca₁₀(PO₄)₆(OH)₂)] and inorganic fullerene-like nanoparticles or inorganic nanotubes; wherein the inorganic fullerene-like nanoparticles or inorganic nanotubes is A₁₋ₓBₓ-chalcogenide where A is a metal or transition metal or an alloy of one metals or transition metals including at least one of the following: Mo, W, Re, Ti, Zr, Hf, Nb, Ta, Pt, Ru, Rh, In, Ga, InS, InSe, GaS, GaSe, WMo, TiW; and B (dopant) is a metal transition metal selected from the following: Si, Nb, Ta, W, Mo, Sc, Y, La, Hf, Ir, Mn, Ru, Re, Os, V, Au, Rh, Pd, Cr, Co, Fe, Ni; x is below or equal 0.003; and the chalcogenide is selected from the S, Se, Te.

2. The composition according to claim 1, wherein the inorganic fullerene-like nanoparticles or inorganic nanotubes are WS₂, MoS₂ or combination thereof.

3. The composition according to claim 1, wherein the concentration of the dopant is below or equal to 0.3 at %.

4. The composition according to claim 1, wherein the composition further comprises brushite, portlandite or combination thereof.

5. The composition according to claim 1, wherein composition is deposited on a substrate forming a film.

6. The composition according to claim 5, wherein the substrate is a biocompatible.

7. The composition according to claim 6, wherein the substrate is a titanium, alloys of titanium, Co—Cr alloys, magnesium, stainless steel, shape memory alloys of nickel-titanium, silver, tantalum, zirconium and novel ceramics or any electrical-conductive substrate.

8. The composition according to claim 7, wherein the titanium is porous.

9. The composition according to claim 1, wherein the concentration of the doped inorganic fullerene-like nanoparticles is between 0.2 wt % to 5 wt % of the composition.

10. The composition according to claim 1, wherein the composition has a positive zeta potential at pH below 6.5.

11. The composition according to claim 1, wherein the composition further comprises a cationic surfactant.

12. The composition according to claim 1, wherein the composition further comprises a polymeric binder.

13. The composition according to claim 5, wherein the film has low friction coefficient of between 0.1 to 0.7.

14. The composition according to claim 13, wherein the low friction is maintained after annealing.

15. A method of coating a metal substrate with the composition according to claim 1, wherein the method comprises electrophoretic deposition plasma spray, ion beam coating, e-beam evaporation, thermal deposition, physical vapor deposition (PVD), aerosol deposition, vacuum deposition, sol gel deposition, or dip coating.

16. The method according to claim 15, wherein the metal substrate is biocompatible.

17. The method according to claim 16, wherein the metal substrate is titanium, alloys of titanium, Co—Cr alloys, magnesium, stainless steel, shape memory alloys of nickel-titanium, silver, tantalum, zirconium and novel ceramics or any electrical-conductive substrate.

18. The method according to claim 15, wherein the metal substrate is anodized prior to the electrophoretic deposition.

19. The method according to claim 15 wherein the electrophoretic deposition is conducted between 2 to 5 hours.

20. A dental or orthopedic implant comprising the composition according to claim 1.

21. A dental or orthopedic implant comprising a film on a biocompatible substrate, wherein the film comprises the composition according to claim 1.

22. A bone regeneration therapy comprising administering an artificial bone implant comprising the composition according to claim 1.

23. A method of osseointegration comprising contacting an artificial bone implant comprising the composition according to claim 1 in a bone needs to be improved.

24. The method according to claim 23, wherein the artificial bone implant comprises a biocompatible substrate coated by a film, wherein the film comprises said composition.

25. The bone regeneration therapy according to claim 22, wherein the artificial bone implant comprises a biocompatible substrate coated by a film, wherein the film comprises said composition.
